(19) United States
(12) Patent Application Publication
(10) Pub. No.: US 2007/0068808 A1
(43) Pub. Date: Mar. 29, 2007

(54) Blood test chip for blood substance measuring device

(75) Inventor: Wei-Jung Lee, Taipei (TW)

Correspondence Address: PATENTTM.US, P. O. BOX 82788, PORTLAND, OR, 97282-0788, US

(73) Assignee: Wei-Jung LEE & Yuan-Chen LEE

(21) Appl. No.: 11/238,577

(22) Filed: Sep. 28, 2005

(51) Int. Cl. G01N 33/487

(52) U.S. Cl. 204/403.01

(57) ABSTRACT

The blood test chip for a blood substance measuring device has an isolated substrate, first and second electrodes, an identification electrode, an electric resistor and a reactive film. When performing a blood test, a blood sample is dropped into an opening on the blood test chip, which is inserted into the device. A microprocessor provides a constant voltage to the second electrode. Two corresponding reaction currents are delivered to a current to voltage converting and amplifying circuit and converted into two corresponding voltage values, which are then delivered to a built-in analog-to-digital converter module of the microprocessor to get two digitized voltage values. A signal read by the identification electrode is compared with the electric current contrast values stored in the built-in memory unit to get an optimum electric current versus concentration function to calculate a concentration value of the test substance in the blood sample.

### BACKGROUND OF THE INVENTION

1. Field of the Invention

The invention relates in general to a blood test chip for a blood substance measuring device, and more particularly to a blood test chip that includes an identification electrode for the blood substance measuring device to automatically select a built-in electric current versus concentration function to perform an operation.

2. Description of the Related Art

Blood tests are very important to monitor people's health, such as in blood-glucose control. With reference to FIG. 1, a first example of a conventional blood substance measuring device 70 includes two slots 711, 712 for receiving a parameter chip 91 and a test chip 90. The test chip 90 is a substrate having two disconnected test electrodes 901, 902. A reaction film (not shown in the diagram) is formed on the two test electrodes 901, 902. Due to differences in manufacturing processes, every lot of test chips has some error value. Hence an adjustment parameter is necessary to make adjustments in every lot of test chips by storing optimum electric current versus concentration functions and test programs on the parameter chip. The same code is then designated to the lot of the test chips and the test parameter chip. When performing a blood test, the test chip cooperates with a parameter chip with the same code when both are inserted into the blood substance measuring device to adjust for the error value.

With reference to FIG. 2, a second example of a conventional blood substance measuring device 80 includes a built-in memory 82, a slot 811 for receiving the test chip 90, and a button 83 for selecting an electric current versus concentration function corresponding to the test chip 90. The memory 82 is used to store multiple electric current versus concentration functions. When performing a blood test, users press the button 83 to manually select the corresponding electric current versus concentration function to get accurate test values.

The aforesaid first conventional blood substance measuring device requires the corresponding parameter chip, which increases manufacturing costs and complexity. Moreover, the second conventional blood substance measuring device requires the user to manually switch the functions, which is rather inconvenient. Hence the conventional blood substance measuring device can be further improved.

### SUMMARY OF THE INVENTION

The present invention provides a blood test chip for a blood substance measuring device. The blood test chip for a blood substance measuring device is configured with an identification electrode to automatically select an optimum electric current versus concentration function for the test chip. In this way, the need for an additional adjustment device or the manual selection of the optimum electric current versus concentration function can be eliminated, so as to simplify the blood substance measuring device and to reduce error.

In order to achieve the above objective, the blood test chip for a blood substance measuring device of the present invention includes an isolated substrate, a first electrode and a second electrode, an identification electrode, an electric resistor and a reactive film.

When performing a blood test, the blood test chip is first inserted into a slot of the blood substance measuring device so that the blood test chip is electrically connected to the blood substance measuring device via a connector. A blood lancet is used to collect a blood sample, and the blood sample is then dropped onto an opening on the blood test chip. After several seconds, a microprocessor provides a constant voltage V to the second electrode via the constant voltage circuit. The voltage V goes through the second electrode, the electric resistor, and the identification electrode to complete a circuit and generate a corresponding reaction current A1 on the identification electrode. The voltage V goes through the second electrode, the reactive film, and the first electrode to complete a circuit and generate a corresponding reaction current A2 on the identification electrode. The two corresponding reaction currents A1 and A2 are delivered to a current to voltage converting and amplifying circuit to be amplified and converted as two corresponding voltage values. The two corresponding voltage values are then delivered to a built-in analog-to-digital converter module of the microprocessor to get two digitized voltage values. A signal that is read by the identification electrode is compared with the different electric current contrast values stored in the built-in memory unit to get an optimum electric current versus concentration function. Then a current signal that is read by the first electrode is substituted for the electric current versus concentration function to calculate a concentration value of the testing substance in the blood sample. Finally the concentration value is displayed on the display screen.

### BRIEF DESCRIPTION OF THE DRAWINGS

FIG. 1 is a perspective view of a conventional blood substance measuring device.

FIG. 2 is a front view of another conventional blood substance measuring device.

FIG. 3 is a perspective view of a blood test chip in accordance with the present invention.

FIG. 4 is a perspective view of the present invention of FIG. 3 when folded.

FIG. 5 is an operational perspective view of the present invention.

FIG. 6 is a functional block diagram of a blood substance measuring device for the blood test chip of the present invention.

FIGS. 7A-7J is a circuit diagram of the blood substance measuring device of FIG. 6.

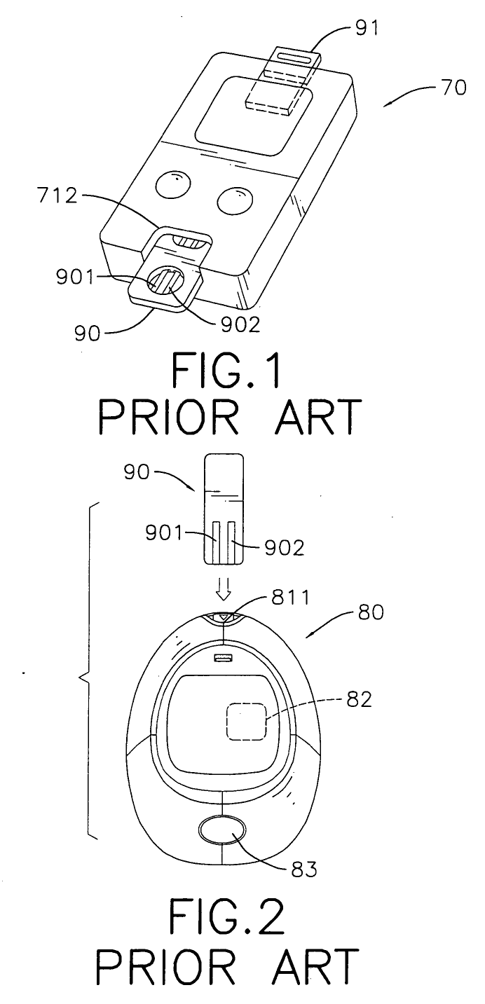

### DETAILED DESCRIPTION OF THE INVENTION

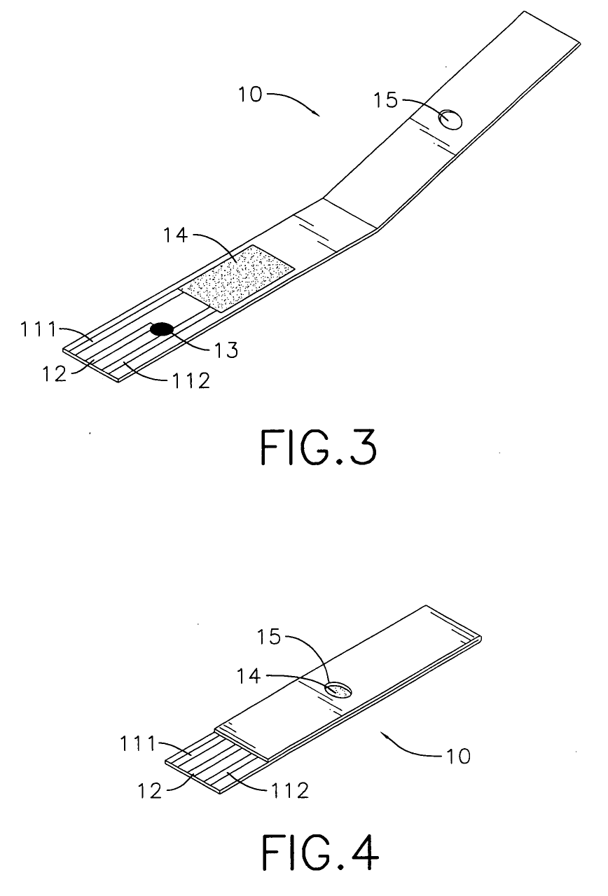

With reference to FIG. 3, a structure of a blood test chip for a blood substance measuring device of the present invention includes a long strip of an isolated substrate 10. Three disconnected electrodes are configured on an end of the isolated substrate 10: a first electrode 111, a second electrode 112, and an identification electrode 12. The second electrode 112 is a reference electrode. A precision electric resistor 13 is configured between the second electrode 112 and the identification electrode 12. The precision electric resistor 13 can have different resistance values in accordance with the manufacturing processes of the blood test chip and the substance to be tested. Furthermore, a reactive film 14 is formed at an appropriate region in a middle part of the substrate 10 and also covers an end of the first electrode 11 and the second electrode 112, so as to make the first electrode 111 and the second electrode 112 electrically connected. Moreover, an opening 15 is further formed on the substrate 10.

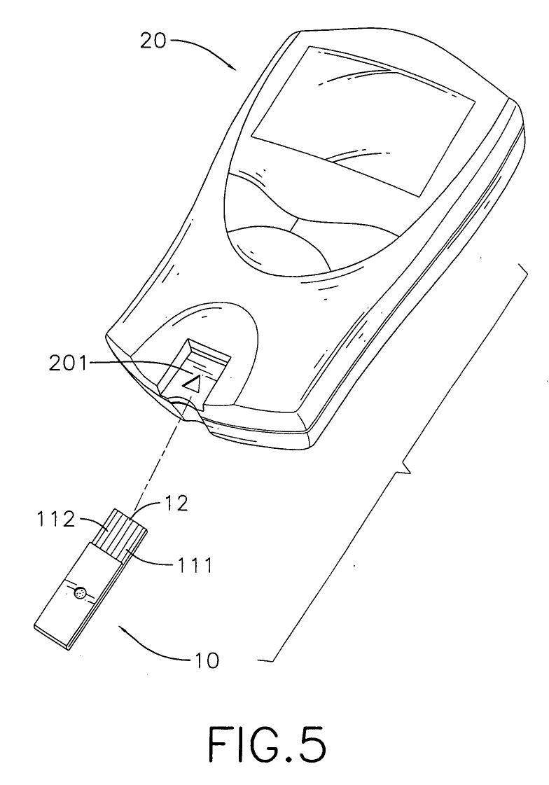

With reference to FIG. 4, when the substrate 10 is folded, an appropriate square measure of the three electrodes is exposed to be electrically connected to a blood substance measuring device. The opening 15 corresponds to the reactive film 14 on the substrate 10, so that blood specimens can drop onto the reactive film 14 via the opening 15 to generate an electrochemical reaction with the reactive film 14.

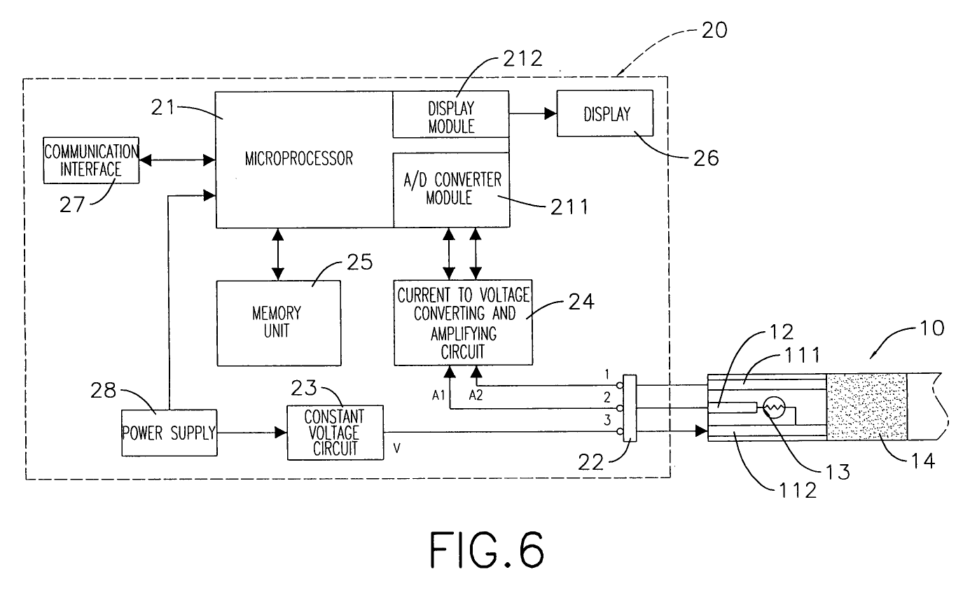

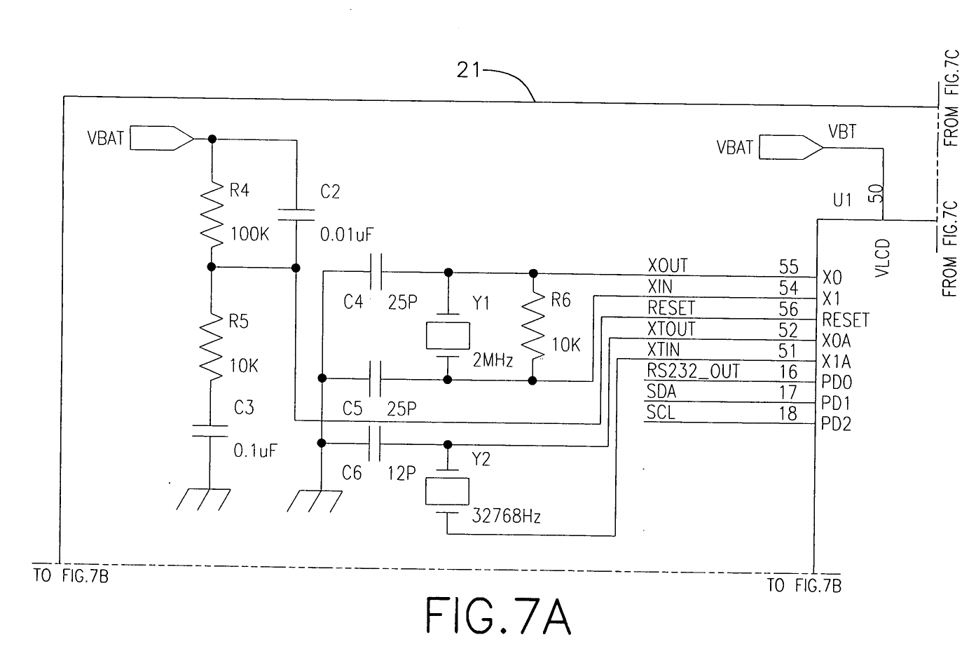

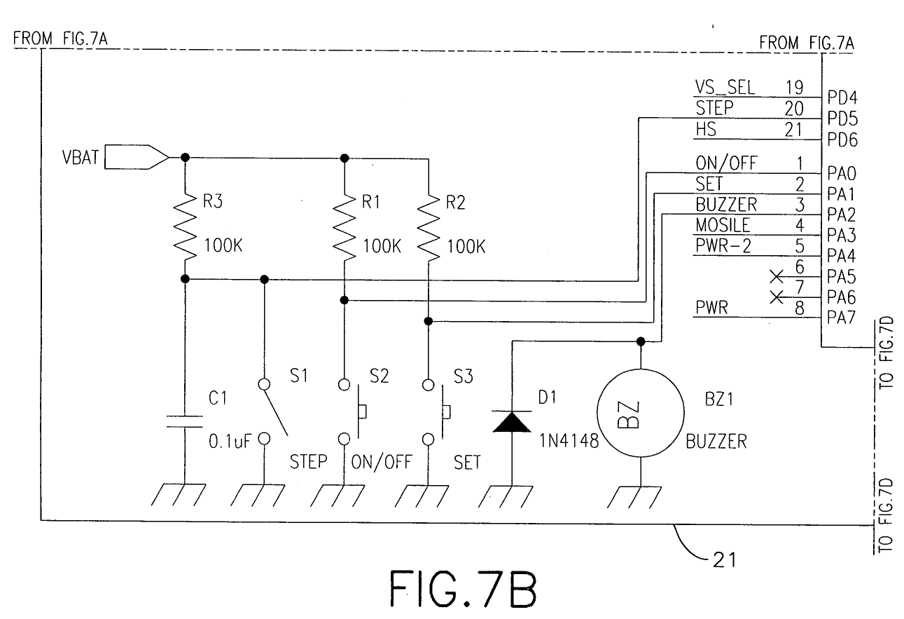

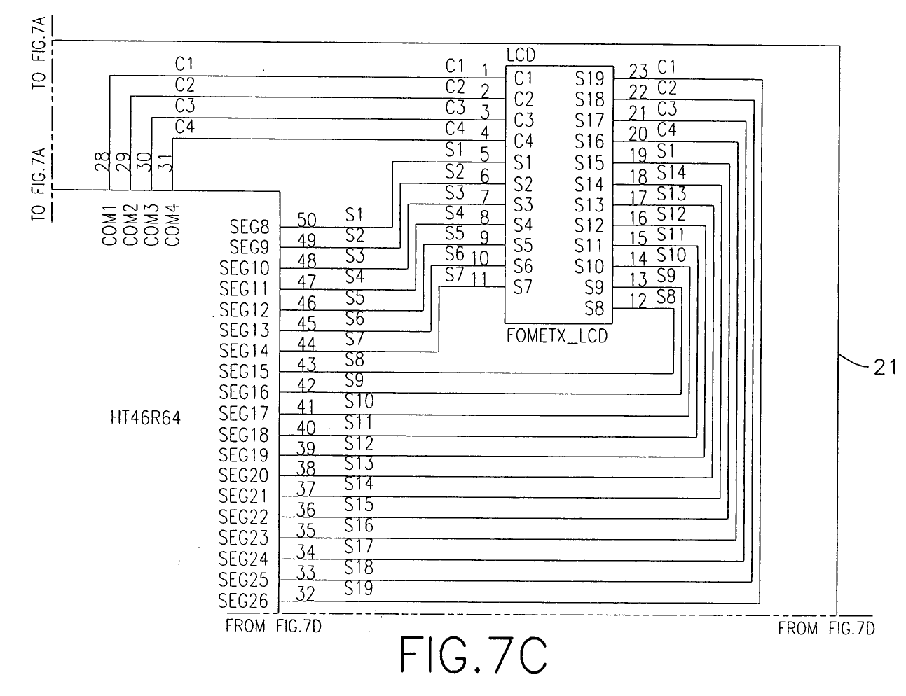

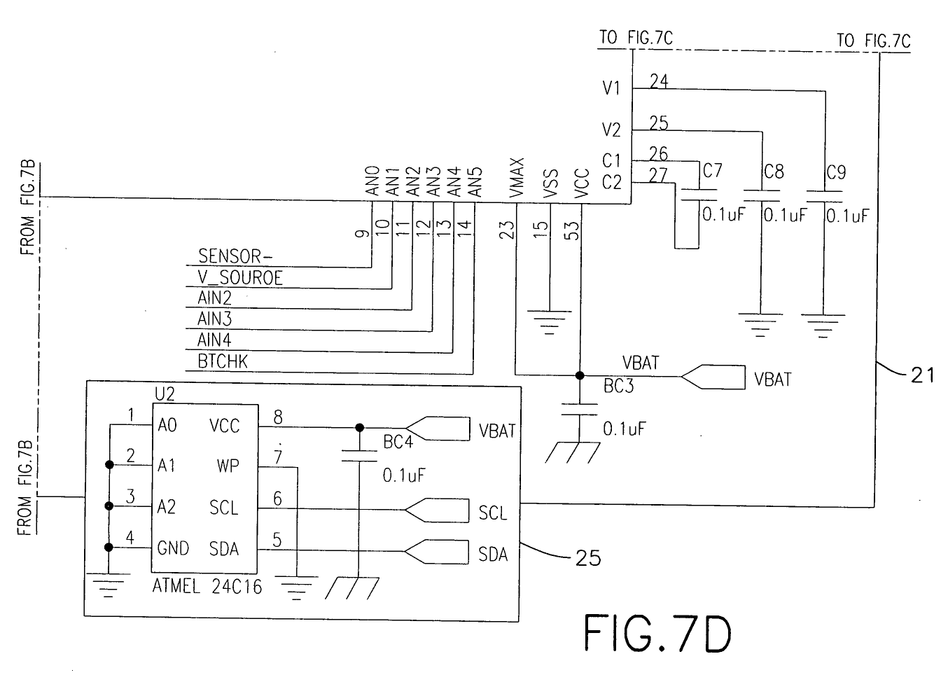

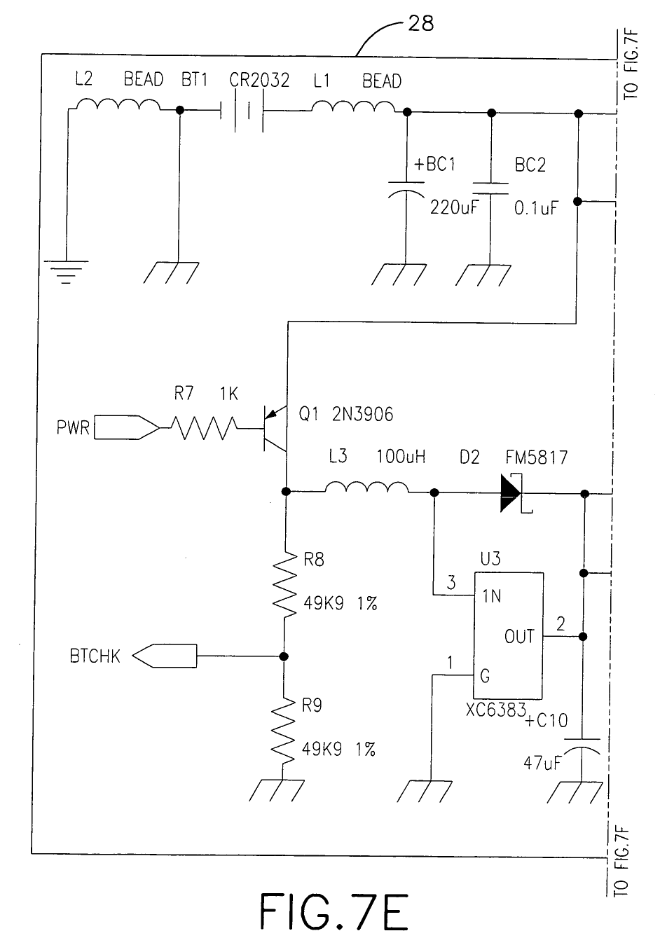

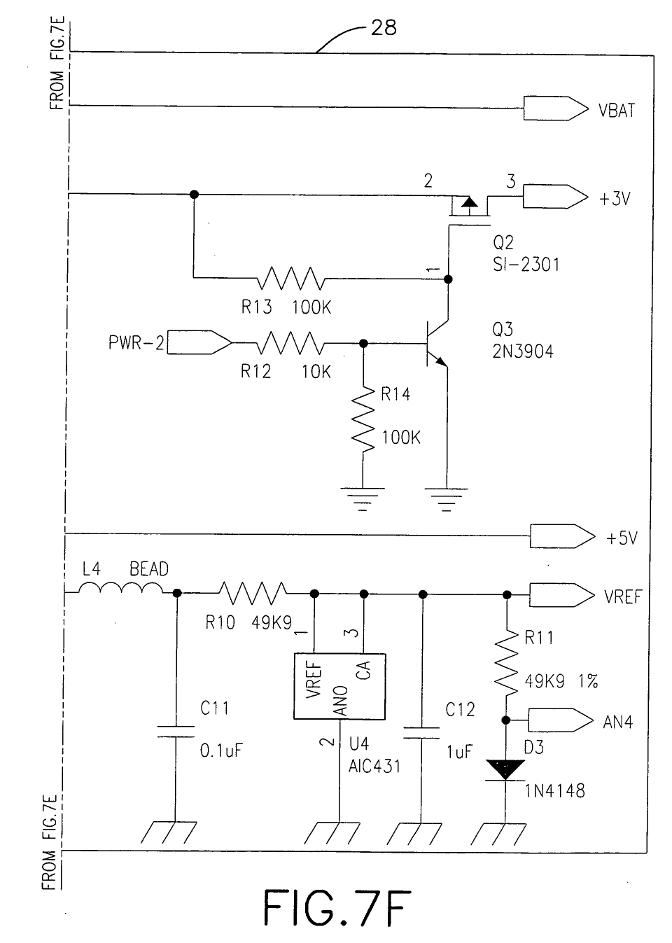

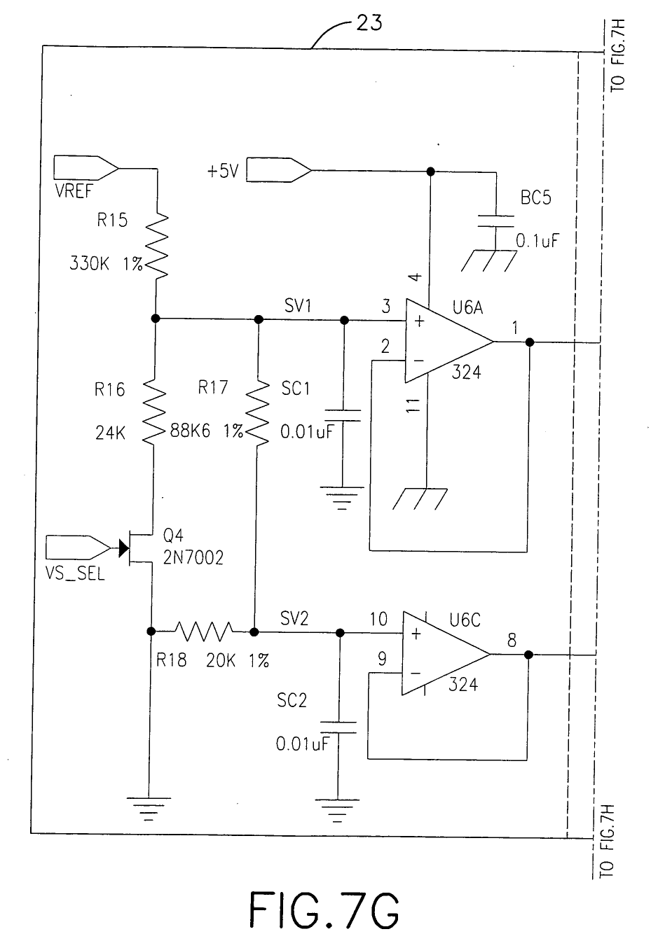

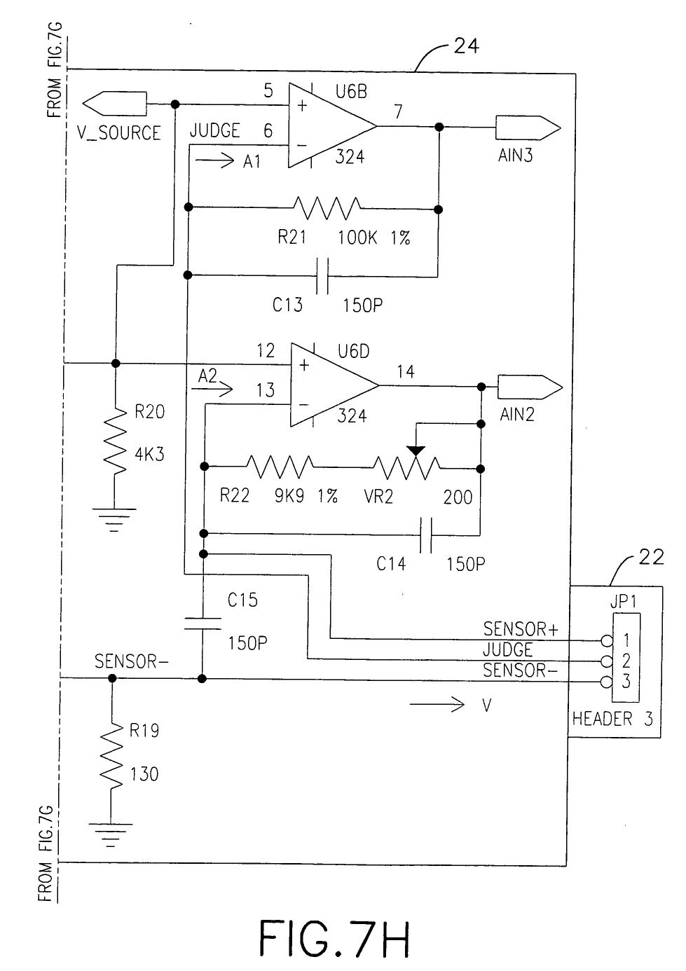

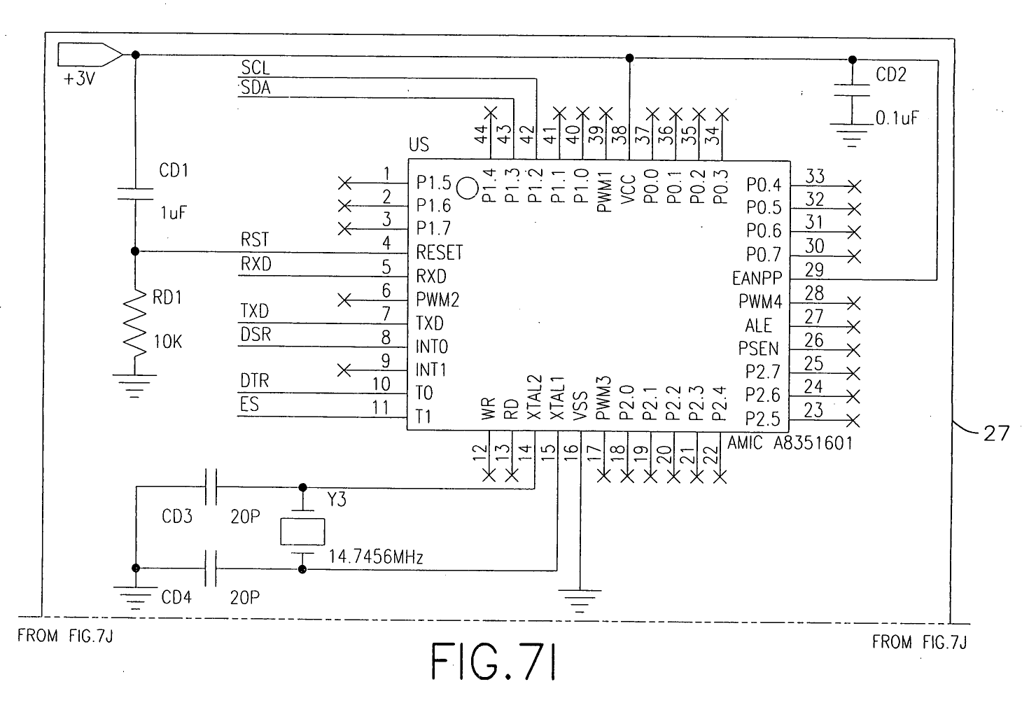

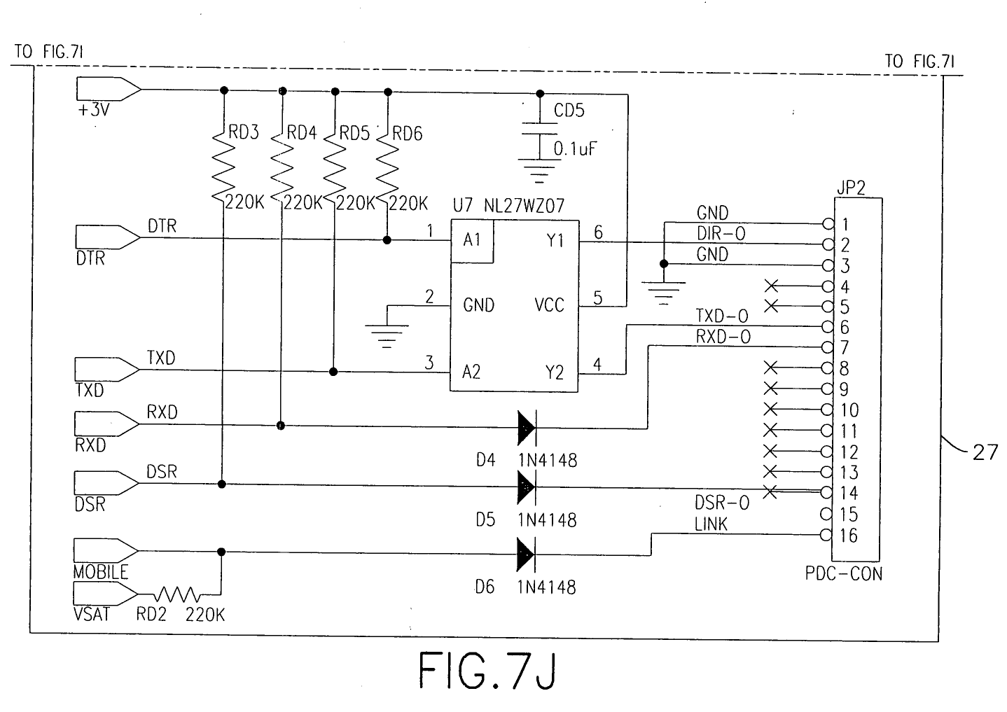

With reference to FIG. 5, a blood substance measuring device 20 includes a slot 201 for holding the blood test chip of the present invention. Moreover, with reference to FIG. 6 and FIGS. 7A-7J simultaneously, the blood substance measuring device 20 further includes a microprocessor 21, a connector 22, a constant voltage circuit 23, a current to voltage converting and amplifying circuit 24, a memory unit 25, a display 26, a communication interface 27, and a power 17 circuit 28.

The microprocessor 21 includes execution procedures of control, examination, adjustment, and analysis. In a preferred embodiment of the present invention, an analog-to-digital converter module 211 and a display module 212 of the HT46R64 microprocessor is used.

Pin 1 of the connector 22 is used to connect the first electrode 111 to the current to voltage converting and amplifying circuit 24. Pin 2 of the connector 22 is used to connect the identification electrode 12 to the current to voltage converting and amplifying circuit 24. Pin 3 of the connector 22 is used to connect the second electrode 112 to the constant voltage circuit 23.

The constant voltage circuit 23 outputs a constant voltage to the second electrode 112 of the blood test chip.

The current to voltage converting and amplifying circuit 24 includes two input terminals to be connected respectively to the first electrode 111 and the identification electrode 12, and also includes an output terminal to be connected to the microprocessor 21.

The memory unit 25 has a built-in a plurality of functions of electric current versus concentration for different substance analyses, which it can provide to the microprocessor 21 and input detector voltage to make a comparison. In the preferred embodiment of the present invention, the memory unit 25 used is an ATMEL 24C16.

The display 26 is connected to an output terminal of the built-in display module 212 of the microprocessor 21 to display related test results.

When the optimum electric current versus concentration function for the test substance analysis is not stored in the microprocessor 21, the communication interface 27 can be connected to an external database to update the required electric current versus concentration function in the microprocessor 21. In the preferred embodiment of the present invention, the communication interface 27 is an RS232 interface.

The power circuit 28 provides the required electricity for the above components to work.

When performing a blood test, the blood test chip is first inserted to the slot 201 of the blood substance measuring device 20, so that the blood test chip is electrically connected to the blood substance measuring device 20 via the connector 22. A blood lancet is used to collect a blood sample, and the blood sample is then dropped into the opening 201 of the blood test chip. After several seconds, the microprocessor 21 provides a constant voltage V to the second electrode 112 via the constant voltage circuit 23. The voltage V goes through the second electrode 112, the precision electric resistor 13, and the identification electrode 12 to complete a circuit and generate a corresponding reaction current A1 on the identification electrode 12. The voltage V goes through the second electrode 112, the reactive film 14, and the first electrode 111 to complete a circuit and generate a corresponding reaction current A2 on the identification electrode 111. The two corresponding reaction currents A1 and A2 are delivered to the current to voltage converting and amplifying circuit 24 to be amplified and converted as two corresponding voltage values. The two corresponding voltage values are then delivered to the built-in analog-to-digital converter module 211 of the microprocessor 21 to get two digitized voltage values. A signal that is read by the identification electrode 12 is compared with the different electric current contrast values stored in the built-in memory unit 25 to get an optimum electric current versus concentration function. Then a current signal that is read by the first electrode 111 is substituted for the electric current versus concentration function to calculate a concentration value of the test substance in the blood sample. Finally the concentration value is displayed on the display 26 via the display module 211.

According to the above-described design, when the blood test chip is inserted to the blood substance measuring device, a separate device to adjust for errors is not required. In addition, users do not need to set or choose any parameters when using the device. Hence the present invention not only reduces inconvenience when in use, but can also save on the cost of a separate device to adjust for errors. The invention also has the characteristics of utility and non-obviousness.

While the invention has been described by way of example and in terms of a preferred embodiment, it is to be understood that the invention is not limited thereto. On the contrary, it is intended to cover various modifications and similar arrangements and procedures, and the scope of the appended claims therefore should be accorded the broadest interpretation so as to encompass all such modifications and similar arrangements and procedures.

What is claimed is:

1. A blood test chip for a blood substance measuring device, the blood test chip comprising:
an isolated substrate;
a first electrode and a second electrode configured on the isolated substrate;
an identification electrode configured on the isolated substrate;
an electric resistor configured between the second electrode and the identification electrode; and
a reactive film formed at a region in a middle part of the substrate and also covering an end of the first electrode and the second electrode.

2. The blood test chip as claimed in claim 1, wherein an opening is formed on the substrate, wherein when the substrate is folded, an appropriate square measure of the three electrodes of the first/second electrodes and the identification electrode is exposed to be electrically connected to the blood substance measuring device, and also wherein the opening corresponds with the reactive film on the substrate.

3. The blood test chip as claimed in claim 1, wherein the electric resistors have different resistance values.

4. The blood test chip as claimed in claim 2, wherein the electric resistor has different resistance values.
